(19) United States
(12) Patent Application Publication
(10) Pub. No.: US 2024/0108433 A1
(43) Pub. Date: Apr. 4, 2024

(54) DEVICES AND METHODS FOR MINIMIZING DAMAGE TO SOFT TISSUE DURING A SURGICAL PROCEDURE

(71) Applicant: Depuy Synthes Products, Inc., Raynham, MA (US)

(72) Inventors: Stephen J. Orphanos, Bridgewater, MA (US); Vivian Liang, South Easton, MA (US); James Brownhill, Norton, MA (US); Mollie Waters, Boston, MA (US); Michael Fortin, Acushnet, MA (US)

(21) Appl. No.: 18/473,574

(22) Filed: Sep. 25, 2023

Related U.S. Application Data
(60) Provisional application No. 63/412,177, filed on Sep. 30, 2022.

(51) Int. Cl. A61B 90/00

(52) U.S. Cl. CPC A61B 90/08; A61B 2090/08021

(57) ABSTRACT

Devices and methods for improved access to a surgical site are the basis of the present disclosure. The improved access can be provided by way of a displacement wrap that can be used to engage tissue that impedes access to the surgical site. The displacement wrap, which can be a textile, can be wrapped around the tissue and subsequently manipulated to move the wrap, and thus the tissue, out of the way to improve access. The displacement wrap includes locking features formed in it that allow for selective tensioning of the tissue being engaged and moved. The locking features can engage another portion of the wrap and/or can be engaged by another component associated with the wrap, such as a locking suture. Methods of using the wrap in various surgical procedures that would benefit from moving tissue to improve surgical site access are also provided.

### CROSS-REFERENCE TO RELATED APPLICATIONS

The present application claims priority to U.S. Provisional Patent Application No. 63/412,177, filed Sep. 30, 2022, the contents of each which is hereby incorporated by reference in its entirety.

### FIELD

The present application relates to devices and methods used in conjunction with shoulder repair procedures, and more particularly relates to a device, often textile, silicone, and/or plastics-based, that can be used to help manipulate soft tissue (e.g., the subscapularis) during surgical procedures like tissue sparing shoulder reconstruction arthroplasties in a manner that minimizes damage to the subscapularis.

### BACKGROUND

Surgical procedures often require that tissue that impedes access to the surgical site be moved or manipulated away from its natural location to gain better access to the surgical site. The access may be to deliver certain instruments or devices to a surgical site in the body, to remove something from the body of the patient, to insert an implant into a prepared bone site, and/or to provide better visualization to the surgeon, among other reasons. In many traditional surgical approaches, the tissue that is in the way may be temporarily detached from one or more natural attachment points to provide the desired access. For example, in traditional surgical approaches in which the subscapularis tendon impedes access to the surgical site, thus requiring the subscapularis be moved to provide better access to the surgical site, the tendon is often removed from its humeral attachment point. Another surgical approach is referred to as a subscapularis tenotomy, which entails mobilizing the subscapularis during a total shoulder arthroplasty.

Alternatively, or additionally, the impeding tissue may be manipulated so that it is out of the way, for example using retractors, metal clamps, a reamer shaft to retract tissues and to manipulate space, sutures, which can be used to mobilize muscles, particularly when they are tenotomized, and/or other instruments and/or tools being held by a surgeon. In a tissue sparing approach in which the subscapularis tendon is in the way but detachment from its humeral attachment point is not desired, the subscapularis will thus remain with its native attachments points. The procedure can then be performed superior and inferior to its tissue borders. This may result, however, in difficult access to the surgical site. If manipulation of the subscapularis or other tissue is employed, the manipulated tissue may be grabbed and pulled out of the way using retractors, those retractors being manipulated and held by the surgeon, by an assistant to the surgeon, and/or by using a device that holds the metal clamp, and thus the tissue gripped by the same, at a fixed location, during the entirety of the procedure. All the while the metal clamp is providing an unnatural, gripping force on the tissue while holding it in an unnatural position, applying tension to the tissue, particularly at the location at which the clamp engages the tissue.

These detachment and manipulation approaches can cause undesirable, and unnecessary, damage to the moved and/or manipulated tissue, as well as tissue or other components in the body (e.g., bone, muscle, other tissue, etc.) that may be attached to the manipulated tissue, in turn increasing healing time after surgery, affecting vascularization of the tissue and/or being detrimental to the function of the impacted tissue and other components in the body.

Accordingly, there is a need to develop devices, systems, and methods that enable tissue, such as a subscapularis tendon, to be easily manipulated or otherwise moved out of the way to provide desired access to a surgical site while also minimizing the amount of forces and other damage to the tissue in the course of such manipulation and movement.

### SUMMARY

The present disclosure is generally directed to various embodiments of a displacement wrap that can be used to non-traumatically engage tissue that impedes access to a surgical site and move the impeding tissue to a location that improves access to the surgical site without causing damage, or at least minimizing damage, to the moved tissue. The wrap can include a textile that can be moved into a locking position to secure the tissue to be moved, and once locked, the tissue can be moved to a location that provides the improved access to the surgical site. The displacement wrap can include an introducer coupled to it, or integrally formed with it, to assist in placing the wrap around the tissue and subsequently moving the wrap and the tissue. The locking features, which can be a plurality of opposed slits for example, can be formed in a portion of the wrap, and then the slits can be engaged to place the displacement wrap in a locked position. The slits that are engaged can adjust the amount of tension applied to the tissue. The slits can be engaged, for example, by another portion of the displacement wrap, such as a portion of the textile having an opening formed therein through which the slits can pass through and selectively engage said portion, or by way of a suture coupled to the displacement wrap, the suture being able to engage the slits and draw displacement wrap towards itself to place it in a locked position. In some embodiments, the wrap can be used to manipulate or move the impeding tissue without the textile being moved into a locking position. For example, the wrap can be positioned under the tissue and used to move the tissue. Various surgical procedures, including but not limited to making humeral cuts, preparing a glenoid preparation for various shoulder procedures, various hip procedures, and/or accessing an implant or a location where an implant is to be disposed, are improved by virtue of the devices and methods disclosed herein.

A surgical device of the present disclosure includes at least one protective portion and one handling portion. The protective portion of the surgical device can be made of any material than can be used to protect the tissue being moved from a surgical site during surgery. The handling portion can include a manipulative structure that can aid in moving, lifting, and/or handling the tissue. The manipulative structure can be, for example, a rigid or stiff structure, or it can be more flexible, such as one or more sutures or suture constructs.

One embodiment of a surgical method includes disposing a displacement wrap around at least a portion of a tissue and placing the displacement wrap in a locked position to hold the tissue in at least one of tension or compression. The method further includes moving the displacement wrap to a displacement location to cause the tissue to move away from its original location to a new location, thereby increasing at least one of access to a surgical site or an available region behind the tissue for a surgical procedure to be performed, and maintaining the displacement wrap at the displacement location, and thus the tissue at the new location, to allow access to at least one of the surgical site or the available region behind the tissue during the surgical procedure. The displacement wrap protects the tissue. The displacement wrap also distributes load across the displacement wrap and the tissue, thus preventing damage to the tissue.

In at least some embodiments, the displacement wrap includes a first portion and a second portion. The first portion can include a first terminal end and a textile, and the second portion can include a second, opposed terminal end. The second, opposed terminal end can be coupled to an introducer. In some such embodiments, the method can further include passing the introducer under the tissue and subsequently sliding the wrap with respect to the tissue to move the textile into contact with the tissue. The action of disposing a displacement wrap around at least a portion of a tissue can include wrapping the textile around at least a portion of the tissue, with the textile being wrapped around at least a portion of the tissue when the displacement wrap is in the locked position. The method can include attaching the introducer to the textile.

In embodiments that include an introducer, the action of placing the displacement wrap in a locked position can include passing the introducer and a first end of the textile coupled to the introducer through an opening formed in a second end of the textile, and engaging at least one locking feature of the first end of the textile with a portion of the second end of the textile, proximate to the opening, to hold the tissue in tension. In embodiments that do not include an introducer, the action of placing the displacement wrap in a locked position can include passing a first end of the textile through an opening formed in a second end of the textile, and engaging at least one locking feature of the first end of the textile with a portion of the second end of the textile, proximate to the opening, to hold the tissue in tension and/or compression.

The action of moving the displacement wrap to a displacement location can further include moving the tissue both superiorly and inferiorly during the surgical procedure without adjusting a positioning of the displacement wrap with respect to the tissue. The tissue can be, in at least some instances, a subscapularis tendon.

The method can also include actions related to unlocking the displacement wrap to adjust a tension and/or compression of the tissue. For example, the method can include unlocking the displacement wrap from the locked position, the locked position being a first locked position, adjusting an amount of tension and/or compression being applied to the tissue, and placing the displacement wrap in a second locked position. The second locked position can be one of a same locked position or a different locked position than the first locked position. The action of adjusting an amount of tension and/or compression being applied to the tissue can include sliding a portion of the displacement wrap with respect to itself. In some such embodiments, in the first locked position, a first locking feature on a first end of the displacement wrap engages a second end of the displacement wrap, and, in the second locked position, a second locking feature on the first end of the displacement wrap engages the second end of the displacement wrap.

The action of maintaining the displacement wrap at the displacement location can further include holding the tissue in tension and/or compression, and attaching a portion of the displacement wrap to an object to maintain the tension and/or compression at the displacement location. In at least some embodiments, the action of placing the displacement wrap in a locked position to hold the tissue in tension and/or compression can further include passing a first portion of the displacement wrap through a loop formed by a locking suture that is coupled to the displacement wrap, engaging at least one locking feature of the first portion of the displacement wrap with the locking suture to hold the tissue in tension, and collapsing the loop against the first portion of the displacement wrap to place the displacement wrap in the locked position.

One embodiment of a device for manipulating tissue during surgery includes a textile having a body with a length and a width, the length being greater than the width. The body includes a first portion and a second portion. The first portion has a plurality of locking features disposed along the length of the textile, and the second portion has a locking opening formed in it. A width of the second portion is greater than a width of the first portion, with the widths of the first and second portions being disposed in a same orientation as the width of the textile. The locking opening of the second portion is configured to receive the first portion such that a tissue receiving opening is formed by at least one of the first and second portions and each locking feature of the plurality of locking features is configured to engage with the second portion, proximate to the locking opening, to place the device in a locked configuration. The locked configuration is one in which the location of the first portion is maintained with respect to the second portion and tissue disposed in the tissue-receiving opening is able to be secured within the tissue-receiving opening. The textile is configured to protect the tissue it engages and distribute load across the textile and the tissue, thus preventing damage to the tissue.

The locking opening can include, for example, a slit formed in the second portion of the body. The plurality of locking features can include, for example, a plurality of first diagonal slits formed along a length of a first side of the first portion of the body, the slits of the plurality of first diagonal slits being substantially parallel to each other, and a plurality of second diagonal slits formed along a length of a second side of the first portion of the body, the slits of the plurality of second diagonal slits being substantially parallel to each other. The second side of the first portion of the body can be opposed to the first side of the first portion of the body and the length of the first and second sides of the first portion of the body can be disposed in a same orientation as the length of the textile.

The textile can include at least one of cotton, nylon, polyester, silk, poly felt, silicone, plastic, rubber, rubber-like materials, biologics, scaffold-type biologics, sutures, filaments, or a combination of any of the same. In some embodiments, the textile can include a cotton material. Regardless of the material, the body can include at least one of a mesh or net structure having a plurality of holes formed in it.

The device can include an introducer. The introducer can be configured to pass into the locking opening of the second portion of the body. In some embodiments, the introducer can be integrally formed with the textile, while in some other embodiments the introducer can be coupled to the textile.

Another embodiment of a device for manipulating tissue during surgery includes a textile having a body with a length and a width, the length being greater than the width. The body includes a first portion and a second portion. The first portion has a plurality of locking features disposed along the length of the textile. The second portion has a locking suture coupled to it. A width of the second portion is greater than a width of the first portion, with the widths of the first and second portions being disposed in a same orientation as the width of the textile. The locking opening of the second portion is configured to receive the first portion such that a tissue receiving opening is formed by at least one of the first and second portions. Each locking feature of the plurality of locking features is configured to engage with the second portion, proximate to the locking opening, to place the device in a locked configuration. The locked configuration is one in which the location of the first portion is maintained with respect to the second portion and tissue disposed in the tissue-receiving opening is able to be secured within the tissue-receiving opening. The textile is configured to protect the tissue it engages and distribute load across the textile and the tissue, thus preventing damage to the tissue.

The plurality of locking features can include, for example, a plurality of first diagonal slits formed along a length of a first side of the first portion of the body, the slits of the plurality of first diagonal slits being substantially parallel to each other, and a plurality of second diagonal slits formed along a length of a second side of the first portion of the body, the slits of the plurality of second diagonal slits being substantially parallel to each other. The second side of the first portion of the body can be opposed to the first side of the first portion of the body and the length of the first and second sides of the first portion of the body can be disposed in a same orientation as the length of the textile.

The textile can include at least one of cotton, nylon, polyester, silk, poly felt, silicone, plastic, rubber, rubber-like materials, biologics, scaffold-type biologics, sutures, filaments, or a combination of any of the same. In some embodiments, the textile can include a cotton material. Regardless of the material, the body can include at least one of a mesh or net structure having a plurality of holes formed in it.

The device can include an introducer. In some embodiments, the introducer can be integrally formed with the first portion of the body of the textile, while in some other embodiments the introducer can be coupled to the first portion of the body of the textile.

### BRIEF DESCRIPTION OF THE DRAWINGS

This disclosure will be more fully understood from the following detailed description taken in conjunction with the accompanying drawings, in which:

FIG. 1 is a perspective view of one embodiment of a displacement wrap in use during a surgical procedure;

FIG. 2 is a top view of another embodiment of a displacement wrap, the displacement wrap being coupled to an introducer;

FIG. 3A is a top view schematic of the displacement wrap of FIG. 2;

FIG. 3B is detailed top view of one terminal end of the displacement wrap of FIG. 3A;

FIG. 3C is a detailed top view of a portion of the displacement wrap of FIG. 3A illustrating a plurality of locking features;

FIG. 3D is a schematic illustration of a design program illustrating a perspective front view of the displacement wrap of FIG. 3A and a window of the design program illustrating various ways by which a geometry and features of the displacement wrap can be adjusted;

FIG. 4A is a perspective view of anatomy of a shoulder, including a subscapularis, the subscapularis impeding access to a surgical site;

FIG. 4B is the perspective view of FIG. 4A with the introducer of FIG. 2 positioned behind the subscapularis;

FIG. 4C is the perspective view of FIG. 4B with the introducer moved with respect to the subscapularis;

FIG. 4D is the perspective view of FIG. 4C with the displacement wrap of FIG. 2 positioned behind the subscapularis;

FIG. 4E is the perspective view of FIG. 4D with the introducer being passed through a locking opening of the displacement wrap;

FIG. 4F is the perspective view of FIG. 4E with a terminal end of the displacement wrap being passed through the locking opening of the displacement wrap;

FIG. 4G is the perspective view of FIG. 4F illustrating locking features of the displacement wrap engaging a portion of the displacement wrap that defines the locking opening while the subscapularis is in a first location;

FIG. 4H is the perspective view of FIG. 4G with the displacement wrap being manipulated to move the subscapularis to a second location;

FIG. 4I is the perspective view of FIG. 4H with the introducer attached to an object in a vicinity of the surgery to keep the subscapularis in the second location;

FIG. 5A is a perspective front view of still another embodiment of a displacement wrap, the displacement wrap being coupled to an introducer that comprises a suture, the displacement wrap also having a locking suture associated therewith;

FIG. 5B is a perspective front view of another embodiment of a displacement wrap, the displacement wrap being coupled to an introducer that comprises a grasper, the displacement wrap also having a locking suture associated therewith;

FIG. 5C is a schematic side view and a schematic front view of the locking suture of FIG. 5B being passed through a hinge;

FIG. 6A is a schematic front view of a portion of yet another embodiment of a displacement wrap, the displacement wrap having a kite wire associated therewith;

FIG. 6B is a schematic side view of the portion of the embodiment of the displacement wrap and the kite wire of FIG. 6A;

FIG. 7A is a front view of another embodiment of a displacement wrap, the displacement wrap comprising a mesh or net structure;

FIG. 7B is a perspective view of still another embodiment of a displacement wrap, the displacement wrap being positioned behind a subscapularis;

FIG. 7C is a perspective view of the displacement wrap and subscapularis of FIG. 7B with a terminal end of the displacement wrap being passed through a locking opening of the displacement wrap;

FIG. 8 is a perspective view of another embodiment of a displacement wrap, the displacement wrap having a button associated therewith;

FIG. 9 is a perspective view of yet another embodiment of a displacement wrap, the displacement wrap having a button associated therewith; and

FIG. 10 is a perspective view of another embodiment of a displacement wrap.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

The figures provided herein are not necessarily to scale, although a person skilled in the art will recognize instances where the figures are to scale and/or what a typical size is when the drawings are not to scale. While in some embodiments movement of one component is described with respect to another, a person skilled in the art will recognize that other movements are possible. Additionally, a number of terms may be used throughout the disclosure interchangeably but will be understood by a person skilled in the art. By way of non-limiting example, the terms “suture,” “filament,” and “suture filament” may be used interchangeably with one another. By way of further non-limiting example, the terms subscapularis, subscapularis tissue, subscapularis tendon, subscapularis muscle, and other variations of the same, may be used interchangeably with one another. Further, to the extent features, sides, or steps are described as being “first” or “second,” such numerical ordering is generally arbitrary, and thus such numbering can be interchangeable. Still further, in the present disclosure, like-numbered components of various embodiments generally have similar features when those components are of a similar nature and/or serve a similar purpose. Lastly, the present disclosure includes some illustrations and descriptions that include prototypes or bench models. A person skilled in the art will recognize how to rely upon the present disclosure to integrate the techniques, systems, devices, and methods provided for into a product in view of the present disclosures.

The present disclosure relates to systems, devices, and methods for preparing a surgical site for surgery. More particularly, embodiments of a surgical device are disclosed herein that can be used to prepare the surgical site, such as a shoulder, by repositioning tissue that impedes access to a surgical site to increase or otherwise improve access to the surgical site. In other words, the present disclosure provides systems, methods, and devices that can aid in a surgical procedure as it pertains to preparing the surgical site while minimizing harm to intervening tissue. As discussed in detail herein, a displacement wrap can be implemented during surgery based on particular needs of the surgery and/or patient. The displacement wrap can include or be a protective portion, also referred to herein as a textile. The protective portion of the displacement wrap can provide a protective shield at a posterior portion of the tissue to protect the tissue, for example, from any unintended cutting from surgical instruments used during surgery (e.g., a saw for performing a humeral head cut). Further, the displacement wrap can be used with a handling portion, also referred to herein as an introducer. In some instances, the introducer may be considered to be part of the displacement wrap, while in other instances the introducer may be considered a separate component that can be selectively coupled to the textile without actually being considered as part of the displacement wrap, such as instances in which the displacement wrap is primarily or solely the textile. The displacement wrap can be used to engage the impeding tissue, for instance by surrounding it (though, as described herein, surrounding does not necessarily have to entail completely surrounding it by wrapping it around 360 degrees of the tissue, though that is an option) and repositioning the engaged tissue to a less-intrusive location, thus improving access to the surgical site. Improved access can include, but is not limited to, better visibility and/or additional size of an opening leading to the surgical site. A person skilled in the art will appreciate other embodiments of displacement wraps beyond those disclosed herein that can be used in conjunction with performing the functions and surgical procedures provided for herein and/or are otherwise derivable from the present disclosures.

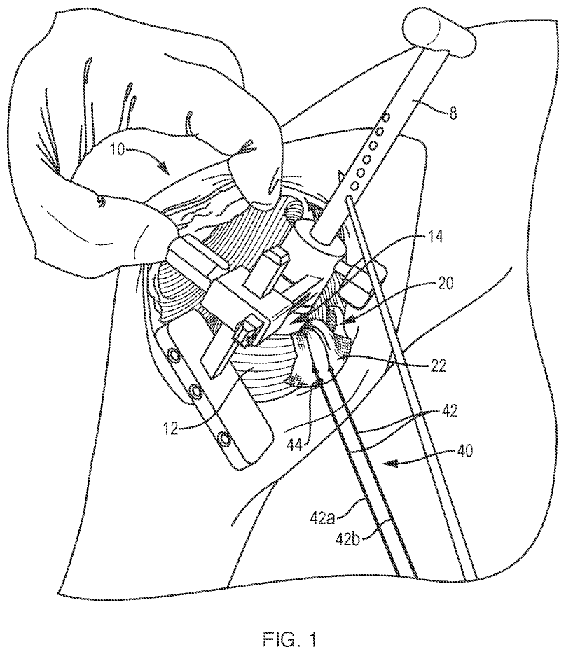

FIG. 1 illustrates one embodiment of a surgical device, referred to herein as a wrapping device or a displacement wrap 20, disposed or utilized to move, lift, or handle tissue, as shown a subscapularis 12, to allow a surgeon or other user improved access to a surgical site 14, as shown a right shoulder 10 of a patient. One non-limiting type of shoulder procedure with which the device 20 can be used is a tissue sparing shoulder reconstruction procedure, with the displacement wrap 20 providing increased visualization of the humeral anatomy, for example inferiorly and/or superiorly. Increased visualization can be provided by compressing surrounding tissues into a smaller volume and gaining better access to surgery site. The compression of the tissue can also enable for easier delivery of instruments or devices to a surgical site in the body, easier removal of something (e.g., tissue, bone growth, undesired cells, etc.) from the body, and/or easier insertion of an implant into a prepared bone site, among other benefits afforded by the displacement wrap 20, and other displacement wraps disclosed herein. In the illustrated embodiment, because the subscapularis tissue 12 impedes access to the site 14, it is moved inferiorly to allow an easier path to the surgical site 14 for a medical instrument 8, as shown a humeral cut guide, and also providing increased visualization for the surgeon and/or others working with the surgeon.

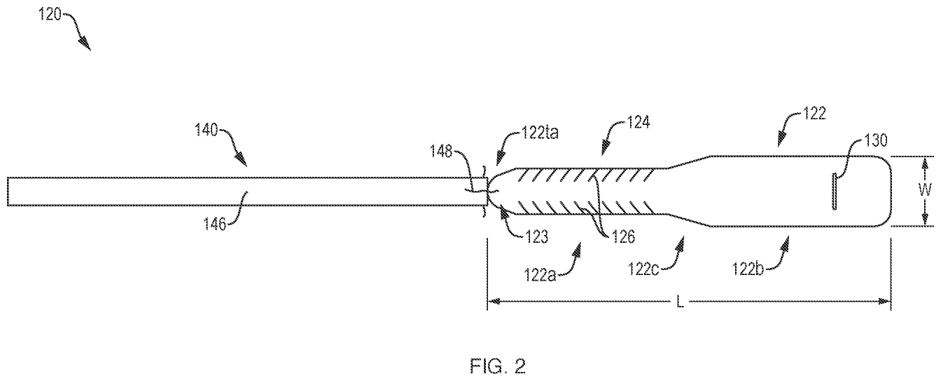

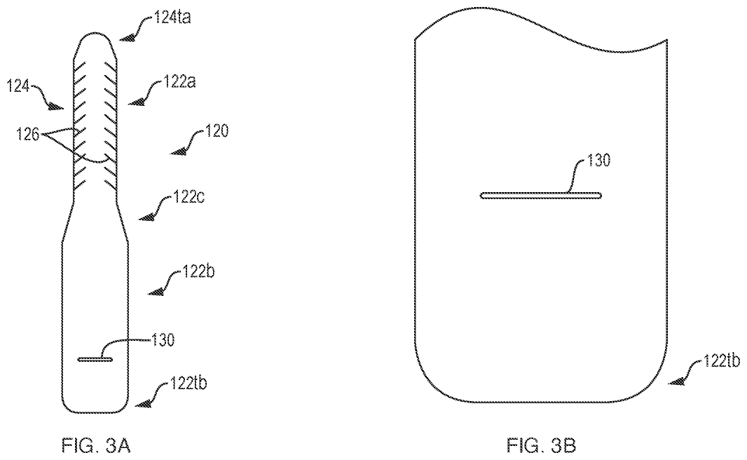

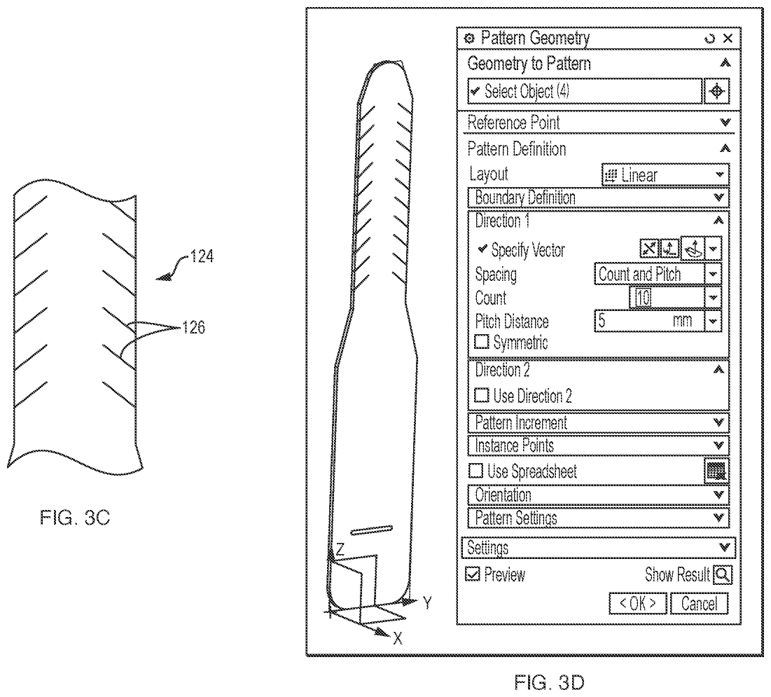

As shown, the displacement wrap 20 comprises a textile 22 that serves as a protective portion of the device 20 (interchangeably referred to as protective portion 22 and/or a body 22 or body portion 22 of the displacement wrap 20). The textile 22 can be, or can be akin to, a tape having a broad, generally flat structure. The surgical textile 22 can often be a rectangular, substantially flat material, pliable such that it can easily be wrapped around tissue to be moved. As used herein, references to a displacement wrap “wrapping around,” being “disposed around,” “encapsulating,” or other similar terms around tissue to be moved does not necessarily require that the displacement wrap be fully (i.e., 360 degrees) around a tissue. Rather, the displacement wrap 20 merely needs to be able to engage a sufficient amount of the tissue to allow the tissue to be moved as desired. The foregoing notwithstanding, it can be beneficial to engage a greater portion of the tissue with the displacement wrap 20 because doing so can help distribute forces that may otherwise be applied to the tissue by a component being used to help move the tissue across the displacement wrap 20, such as an introducer 40, instead of across the tissue. In the illustrated embodiment, the surface area of the surgical textile 22 provides a large surface area upon which forces imparted on the surgical textile 22 by way of the introducer 40 can be spread across, thus minimizing the potential for damage to tissue engaged by the surgical textile 22. Accordingly, while fully encapsulating tissue with the displacement wrap 20, or other displacement wraps provided for herein or otherwise derivable from the present disclosures, is not required, doing so may be beneficial to the extent it increases a surface area across which forces imparted on the displacement wrap 20 can be spread. Alternative embodiments, and further details and benefits of displacement wraps, are provided herein with respect to, for example, a displacement wrap 120 of FIGS. 2-3D.

The displacement wrap 20 can be used in conjunction with a component configured to help manipulate or position the displacement wrap 20 to a desired position, among other features. This component may be referred to, among other names, an introducer or handling portion 40. Together, the displacement wrap 20 and the introducer 40 can aid in moving, lifting, and/or handling the tissue 12. The introducer 40 itself can help direct the displacement wrap 20, and thus tissue associated therewith, to desired locations, and it can also help provide tension or compression to hold the desired location of the displacement wrap 20, and thus the tissue associated therewith. In the illustrated embodiment, the introducer 40 is a suture or filament 42 and is a separate component from the displacement wrap 20. The suture 42 can be coupled to the textile 22 using any known technique for associating suture with another component, such as tying one or more knots in the suture to couple or otherwise associate the suture 42 with the textile 22. The attachment can be substantially rigid such that little or no movement exists between the suture 42 and the textile 22, or alternatively, the attachment can be more fluid, thus allowing the textile 22 to slide along at least a portion of the suture 42, creating a moveable attachment between the two components. By way of non-limiting example, in the illustrated embodiment the suture 42 comprises a single suture having two limbs 42a, 42b, the suture 42 having a sliding knot 44 formed therein such that the suture 42 forms a loop, the loop defining an opening. Such a configuration can allow tension to be increased and eased as desired in conjunction with manipulating the displacement wrap 20, and thus the tissue 12 associated therewith, utilizing what is sometimes referred to in the art as a zip-tie and/or ratcheting approach. Tension adjustment may occur, for example, based on the way the patient's body responds to the tension being applied. One or more half-hitch knots (not shown) can be formed to selectively lock the displacement wrap 20, and thus the tissue 12 associated therewith, at a desired location during the surgical procedure, with such knots being able to be untied and re-tied if the displacement wrap 20, and thus the tissue 12 associated therewith, is to be moved and held and multiple locations during the procedure. In alternative embodiments, pre-tied knots with self-locking features can be employed in conjunction with and/or in lieu of the sliding knot 44 and/or half-hitch knots. Further teachings related to these types of configurations are disclosed at least in U.S. Pat. Nos. 9,060,763, 9,271,716, and 9,345,468, the contents of each which is incorporated by reference herein in its entirety. A person skilled in the art, in view of the present disclosures, would understand how to apply the concepts of the patent incorporated by reference into attaching and manipulating such suture constructs with the displacement wrap 20 in view of the present disclosures.

Most any suture is suitable for use with the displacement wrap 20. In the illustrated embodiment, the suture 42 is a PROKNOT™ suture available from DePuy Synthes (Raynham, MA). A person skilled in the art will appreciate other materials, objects, etc. that can be used as an introducer, some of which are provided for herein and described in greater detail with respect to FIG. 2.

The displacement wrap 20 can be utilized independently or in conjunction with one or more other surgical instruments being used to perform a surgical procedure, e.g., the instrument 8. The user can utilize the displacement wrap 20 by grabbing it with his or her fingers or hand. Alternatively, or additionally, the user can hold the displacement wrap 20 with some other device or tool that can aid in manipulating and/or moving the displacement wrap 20, such as metal clamps (not shown) and/or the suture 42. In the embodiment of FIG. 1, the suture 42 can be used to apply tension to the displacement wrap 20 to move it, and the tissue 12, inferiorly. To maintain the inferior location of the displacement wrap 20 and tissue 12, continuous tension can be applied by the suture 42, for instance by a user and/or by tacking the suture in a manner that allows for the continuous application of tension without requiring a user to supply that tension. As noted above, in at least some embodiments, the amount of tension applied to the displacement wrap 20 can be changed during the course of the surgical procedure, for instance by manipulating the suture 42, e.g., sliding it, with respect to the displacement wrap 20 and holding it at various tensions and/or locations throughout the course of a procedure.

As noted above, the displacement wrap 20 can, but does not have, to be fully wrapped around and/or fully encapsulate the tissue, where fully wrapping can include coverage around the tissue 12 that is at or nearly at 100% with about 360 degrees of coverage. More specifically, the displacement wrap 20 can be disposed to partially wrap or cover the tissue 12 (e.g., coverage around of the tissue 12 can be less than 100% with less than 360 degrees coverage). For example, the displacement wrap 20 can be disposed to have coverage of the tissue approximately in the range of about 10 degrees to about 350 degrees, or any other suitable range therebetween (e.g., allowing for coverage about 20 degrees around the tissue, about 30 degrees around the tissue . . . through about 350 degrees around the tissue without thus being fully wrapped around the tissue). Alternatively, or additionally, the displacement wrap 20 can be passed under the tissue 12 and be used to move or manipulate the tissue 12, without necessarily fully wrapping the tissue 12. For example edges or ends of the displacement wrap 20 can be repositioned to move or manipulate the tissue 12. As provided for herein, to the extent descriptions indicate that any displacement wrap of the present disclosure is disposed around (or other similar terms) the tissue, these other, not full 360 degree coverage, are encompassed by such descriptions unless explicitly stated that 360, or near 360 degree coverage, is desired in a particular instance. Likewise, the present disclosure also contemplates instances where the displacement wrap 20, or other wraps provided for herein or otherwise derivable from the present disclosures, can be wrapped fully around the tissue and then continue to be at least partially, or fully around, the tissue one or more additional times, i.e., permitting the wrap to provide greater than 360 degree coverage.

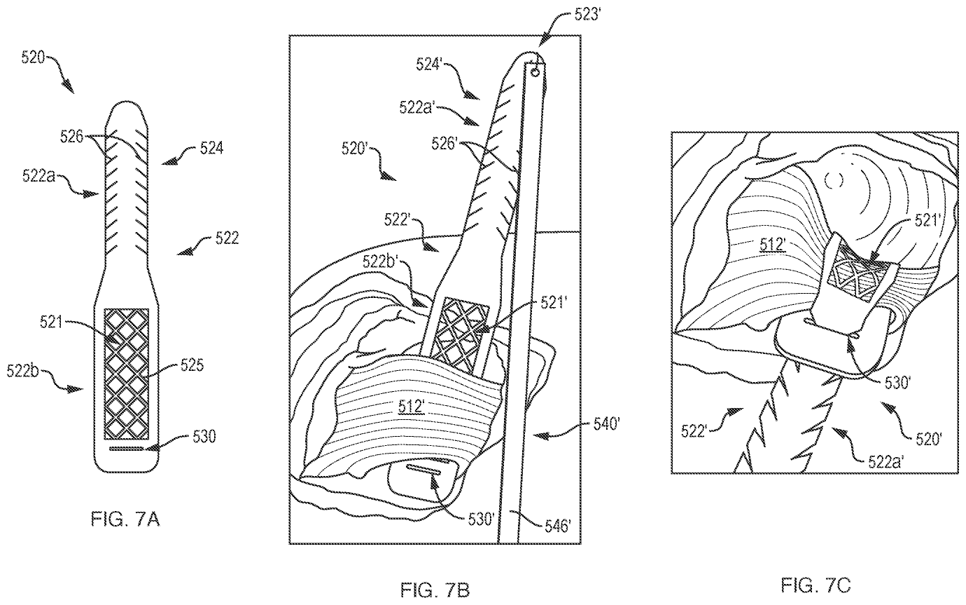

FIG. 2-3D provides for another embodiment of a displacement wrap 120. Similar to the displacement wrap 20, the displacement wrap 120 includes a protective portion 122, also referred to as a body or body portion. In this instance, the protective portion 122 comprises a textile, and is thus also referred to as a textile 122. Any number of materials can be used to form the textile 122, including but not limited to cotton, nylon, polyester, silk, poly felt, silicone, plastic, rubber, rubber-like materials, biologics, scaffold-type biologics, sutures/filaments (such as in a weave configuration as described below with respect to FIGS. 7A-7C), and/or a combination thereof, and the material can be stretchable, or alternatively, un-stretchable material. In some embodiments, additive coatings or other materials can be added to the textile 122. The additive coatings can include one or more anti-infective, anti-fungal, anti-bacterial, anti-microbial, and/or anti-viral material to, for example, prevent infection during surgery, reduce friction on the tissue 12, and/or otherwise enhance the performance of the displacement wrap 120. As with the displacement wrap 20, the displacement wrap 120 can more generally be a component designed to engage the tissue in a non-invasive manner, such as be wrapping around at least a portion of it, up to and including fully wrapping around it, and thus minimizing any damage to the tissue and spreading forces applied to it across a surface area of the body 122.

The textile 122 can have a variety of shapes and sizes. Generally it can be described as having a length L, a width W (labeled for a portion of the textile, but the term can be used in conjunction with the varying widths illustrated across the length L), and a thickness (not shown, but into and out of the page in FIG. 2), with the length L being a largest dimension, the width W being the second-largest dimension, and the thickness being the smallest dimension. As shown in FIG. 2, the width W of the textile can change across the length, with the textile having multiple portions with different widths. In the illustrated embodiment, there is a first portion 122a, a second portion 122b, and a connecting or transition portion 122c, with the first portion 122a having the smallest width, the second portion 122b having the largest width, and the transition portion 122c having a width that gets gradually larger as it moves from the first portion 122a to the second portion 122b. Further, the first portion 122a includes a tapered terminal end 122ta that provides for an even smaller width than a main portion of the first portion 122a. In some embodiments, the first portion 122a can include an introducer receiver 123 formed therein for receiving an introducer and/or a component that couples the introducer to the textile 122, as shown a suture 148.

The length L of the textile 122 can be approximately in the range of about 50 mm to about 200 mm, including any length or range of lengths comprised therein. The width W of the first portion 122a, the second portions 122b, and the transition portion 122c can be approximately in the range of about 2 mm to about 15 mm, including any width or range of widths comprised therein, with the width W of the first portion 122a typically being smaller than the width W of the second portion 122b, and the width W of the transition portion 122c gradually changing as it moves from the width W of the first portion 122a to the width W of the second portion 122b. The width of the first portion 122a may be determined to enable optimal load distribution when the displacement wrap 120 wraps tissue. A thickness of the textile 122 can be approximately in the range of about 0.1 mm to about 2.0 mm, including any thickness or range of thicknesses comprised therein. As noted earlier, the length L is typically greater than the width W. For example, the length L of the textile 122 can be approximately in the range of about 5% to about 40% longer than its width, or approximately in the range of about 40% to about 60% longer than its width, or approximately in the range of about 60% to about 80% longer than its width. Other percentages are possible.

The first portion 122a can include a plurality of locking features or structures 124 designed to help secure the displacement wrap 12 around at least a portion of tissue. In the illustrated embodiment the locking features 124 comprise a plurality of diagonally disposed corrugations or slits 126 formed along a portion of the length L of the body 122 of the displacement wrap 120. As shown, the slits 126 are formed on opposed sides or edges of the width W of the first portion 122a, forming two rows of diagonally-disposed slits. In the illustrated embodiment, each row includes 10 slits, though more or less slits are possible. The slits 126 can typically be aligned with each other on the opposed sides, though they do not necessarily have to be disposed as such. Likewise, while in the illustrated embodiment the slits 126 are substantially spaced equally from each other along the length L of the textile 122, substantially parallel to each other, they do not have to be disposed as such. The locking features 124 can be disposed along any portion of the length L of the body 122, or more particularly along the length of the first portion 122a. A person skilled in the art will appreciate that the more length that is covered by the locking features 124, the greater the range of tissue size that can be captured by the displacement wrap 120 because it provides for additional diameters that can be formed by the locked displacement wrap 120. Likewise, the greater number of slits 126 formed provide for a greater variation in options of diameters that can be formed by the locked displacement wrap 120. A diameter formed by the locked displacement wrap 120, as illustrated in greater detail below, is a diameter formed when the displacement wrap 120 is disposed in a locked position to engage the tissue and subsequently move the tissue to a desired location. Tissue held by the displacement wrap 120 in the locked position can hold the tissue in tension and/or can compress the tissue to a desired location, thus improving access to the surgical site. By way of non-limiting examples, in some embodiments a length of the first portion 122a across which the locking features 124 are formed constitutes approximately 20% of a length of the first portion 122a to approximately 80% of a length of the first portion 122a, or approximately 40% of a length of the first portion 122a to approximately 60% of a length of the first portion 122a, including any percentage or range of percentages comprised therein. In some embodiments, the locking features 124 may occupy more than about 70% of the first portion 122a.

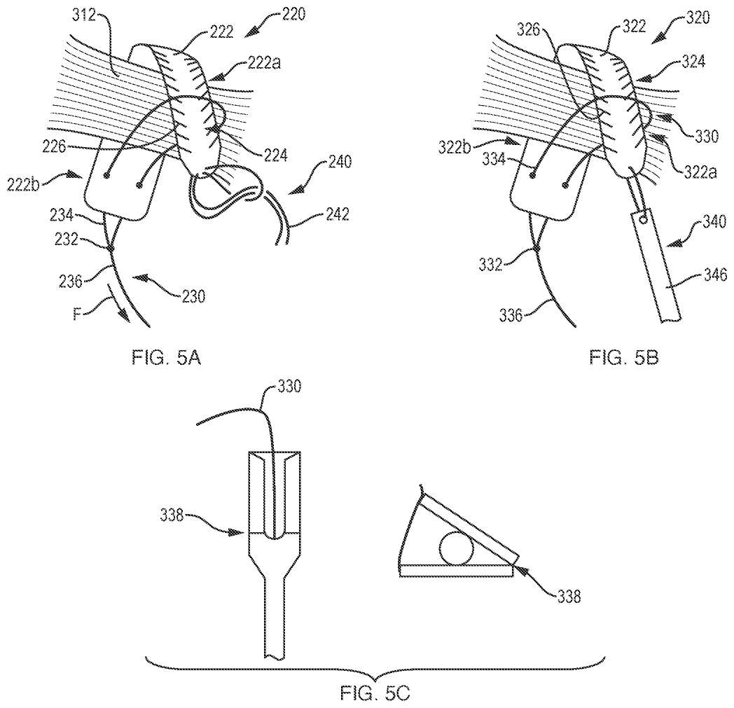

As described in greater detail below, the body 122 in which the slits 126 are formed can be configured to engage another portion of the body 122, proximate to a locking opening 130, such that the textile 122 can be secured in a position that engages and holds tissue for subsequent manipulation. Relatedly, the terminal end 122ta of the first portion 122a can be tapered as shown, which can help the first portion 122a pass into and through the locking opening 130. A person skilled in the art will appreciate other locking features that can be used in addition to, or in lieu of, the slits 126, including but not limited to the locking features provided for with respect to FIGS. 5A-10.

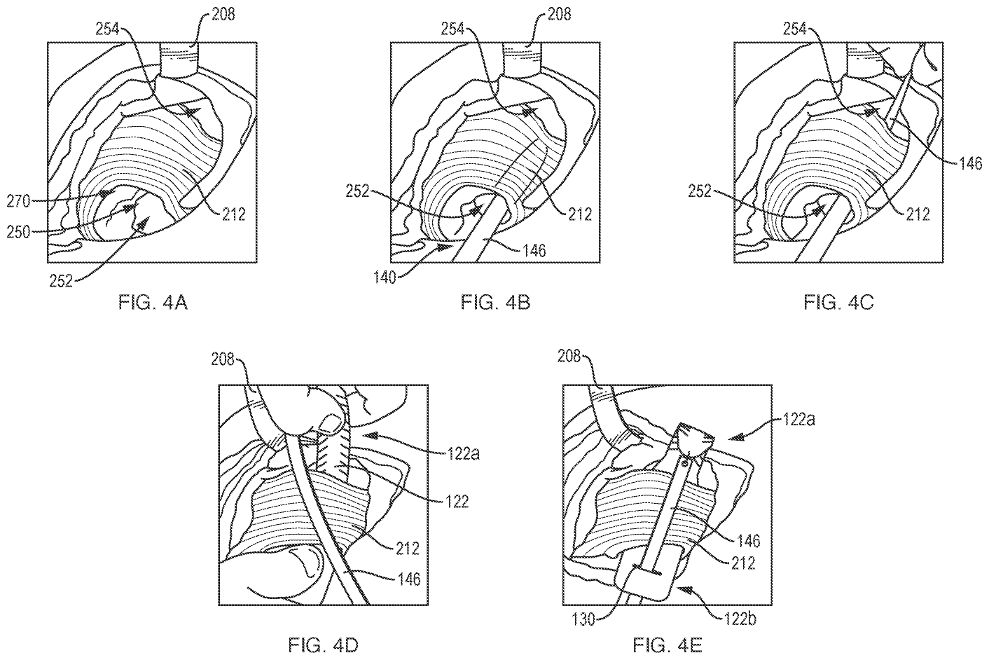

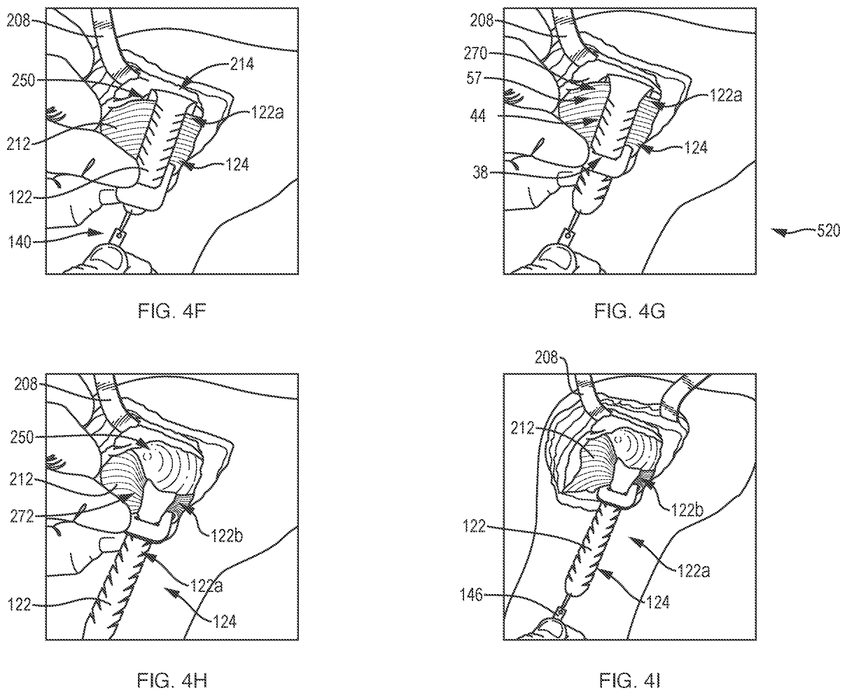

The second portion 122b can include the locking opening 130, which can be can be a slit, a hole, or any space sized to allow an introducer 140 and the first portion 122a of the textile 122 to pass through it. The locking opening 130 can be designed, positioned, or configured in any known manner, but as shown it is disposed substantially centrally with respect to the width W of the body 120, and is disposed more closely to a terminal end 122tb of the second portion 122b than the transition portion 122c. The locking opening 130 can receive first portion 122a and the portion of the second portion 122b that forms the locking opening 130 can be engaged by the locking features 124 to secure a position of the first portion 122a with respect to the second portion 122b to create a tissue opening 57 that can receive a tissue therein, for instance by virtue of the textile 122 being wrapped around the tissue (as shown in FIG. 4H). Each locking feature, e.g., slit 126, of the plurality of locking features 124 can be configured to engage with the second portion 122b to create the tissue opening 57 and to place the displacement wrap 120 in a locked position. When the displacement wrap 120 is in a locked position, the location of the first textile portion 122a can maintained with respect to the second textile portion 122b and the tissue 12 can be secured in the tissue opening 57, in tension and/or compression. Further, while the terminal end 122tb is shown as having curved edges, in other instances it may not be curved. Further, in at least some embodiments, the displacement wrap 120 can have rounded or circular features formed thereon, such as on edges that define the width of the displacement wrap 120 and that engage tissue when the displacement wrap 120 is wrapped around tissue, to help cradle the soft tissue in a manner that minimizes or avoids damage to the tissue.

The first portion 122a can be connected to the second portion 122b by the tapered connecting or transition portion 122c. The transition portion 122c allows the locking portion of the displacement wrap 120 to be integrated with the main body and/or protective portion of the displacement wrap 120. The overall design is such that the displacement wrap provides for a low profile, thus minimizing the potential for the wrap 120 to interfere with and/or damage surrounding tissue and the like.

FIGS. 3A-3C provide further details and illustrations of the displacement wrap 120, including some illustrations of non-limiting exemplary dimensions that can be associated with the same. For example, as shown, a length of the first portion 122a can be about 65 mm and a length of the second portion 122b can be about 65 mm. The length of the first portion 122a can be the same as, more than, or less than the length of the second portion 122b. As also shown, a length of each diagonal slit 126 can be about 10 mm. In other embodiments, the length of each of the slits 126 can be approximately in the range of about 5 mm to about 10 mm, or approximately in the range of about 10 mm to about 20 mm, or approximately in the range of about 20 mm to about 30 mm, including any length or range comprised therein, as well as other lengths. A person skilled in the art will appreciate that the angle and length of the slits 126, the distance between two consecutive slits 126, and/or the number of slits 126 in the first portion 122a can affect the performance and functionality of the displacement wrap 120, and it is within the skill of the art to optimize such dimensions related to the slits 126 in view of the present disclosures.

As shown, the locking opening 130 can be a slit with a width of about 0.5 mm in one embodiment. In other embodiments, the width of the locking opening 130 can be approximately in the range of about 0.2 mm to about 3 mm, or approximately in the range of about 3 mm to about 5 mm, including any width or range of widths comprised therein. As also shown, a length of the locking opening 130 can be about 12 mm in one embodiment. In other embodiments, the length of the locking opening 130 can be approximately in the range of about 1 mm to about 5 mm, or approximately in the range of about 5 mm to about 12 mm, including any length or range of lengths comprised therein. Still further, as also shown, the locking opening 130 can be located about 20 mm from the terminal end 122tb. In other embodiments, the locking opening 130 can be located approximately in the range of about 10 mm to about 20 mm from the terminal end 122tb, or approximately in the range of about 20 mm to about 30 mm from the terminal end 122tb, or approximately in the range of about 30 mm to about 40 mm from the terminal end 122tb.

FIG. 3D provides a schematic illustration of a design program that provides some non-limiting examples of various geometries and features of the textile 122 that can be adjusted. In the illustrated embodiment, the features of the textile 122 that are able to be adjusted are the slits 126. Various parameters that can be altered include, but are not limited to, the number of slits 126, a shape of the slits 126, a pitch of the slits 126, a distance between the slits 126, and angle of the slits 126, and/or a location of the slits 126 with respect to the textile 122. Such a design program can also be used to adjust the various parameters provided for herein related to the displacement wrap 120, the textile 120, and/or an introducer 140, among other components and features disclosed herein or otherwise derivable from the present disclosures.

Turning back to FIG. 2, similar to the displacement wrap 20, the displacement wrap 120 can be used in conjunction with, or alternatively can include, the introducer 140. The introducer 140 illustrated in FIG. 2 is a more rigid grasper 146 than the suture 42 of the introducer 40. The rigid grasper 146 can be, by way of non-limiting example, a plastic tab, a zip tie, any other rigid or semi-rigid material, and/or materials such as plastic, metallic, rubber, cloth, suture (e.g., the introducer 40), and/or a combination thereof. The introducer 140 can have a higher rigidity than the textile 122. In some embodiments, the introducer 140 can include the same material as the textile 122, but may be chemically treated to have a higher rigidity than the textile 122. Also by way of non-limiting examples, the introducer 140 can include a hook, a clip, a zip tie, a thread, or a band, among other features that can help engage the introducer 140 with the displacement wrap 120. Alternatively, or additionally, a distal end of the introducer 140 can include a device or feature (e.g., tie, hook, button, buckle, hook-and-loop fastener, etc.) to allow attachment and/or securement of the displacement wrap 120 to an external object being used during surgery (e.g., a surgical instrument, a patient drape, etc.). This can allow for tensioning to be held in conjunction with the surgical procedure. This action can more generally be described as applying a vector load to the wrap 120, and as provided for herein, another instrument or the like can be used to secure the displacement wrap at this displacement location, for at least a portion of the surgical procedure where increased access and/or visualization is desired. A person skilled in the art will appreciate many mechanisms or devices that can be designed, configured, and/or implemented to couple the introducer 140 to one or more external objects during a surgical procedure. In the illustrated embodiment, a suture 148 couples the introducer 140 to the displacement wrap 120 by passing through the introducer receiver 123 (alternatively, the suture 148 can pass through the displacement wrap 120 without having a pre-formed opening like the introducer receiver 123), although a person skilled in the art will appreciate many other mechanisms or devices that can be designed, configured, and/or implemented to couple the introducer 140 to the displacement wrap 120 and/or another object nearby, as provided for above. While in the illustrated embodiment the textile 122 and the introducer 140 are separate components, in other embodiments the textile 122 and the introducer 140 can be molded or immovably attached to form one unit or component that comprises the displacement wrap 120, e.g., the displacement wrap 120 can comprise both the textile 122 and the introducer 140.

Similar to the introducer 40, the introducer 140 and displacement wrap 120 can aid in moving, lifting, and/or handling tissue associated with the displacement wrap 120. The introducer 140 itself can help direct the displacement wrap 120, and thus tissue associated therewith, to desired locations, and it can also help provide tension to hold the desired location of the displacement wrap 120, and thus the tissue associated therewith. The length of the introducer 140 can be approximately in the range of about 20 mm to about 200 mm, including any length or range of length comprised therein, while the width of the introducer 140 can be approximately in the range of about 2 mm to about 20 mm, including any length or range of length comprised therein.

Capabilities such as those described above with respect to the introducer 40, e.g., adjusting a tension multiple times during the course of a surgical procedure, can be applied to the introducer 140 as well. Further, while in many instances the devices provided for herein, such as the displacement wraps 20, 120, and others provided for herein, are intended for single patient use and can be disposed of after use, one or more components provided for herein, such as the displacement wrap 120 and/or the introducer 140, can be re-usable upon proper sterilization of the same where appropriate.

A kit can be formed from the various components described herein. For example, one or more displacement wraps having a configuration of one or both of the displacement wraps 20, 120, and/or other displacement wraps provided for herein or otherwise derivable from the present disclosures, can be provided as part of a kit. Different displacement wraps of the kit can be provided for different purposes or uses and/or multiple types of the same displacement wrap can be provided to provide replacements or to provide multiple of the same type for the same surgical procedure if multiple tissue displacements would be helpful. The different displacement wraps provided can be suitable for different types of procedures, different patient anatomies, and/or different surgeon preferences, among other reasons for having a kit that comprises multiple displacement wraps. Likewise, multiple introducers, such as the introducers 40, 140, and/or other introducers provided for herein or otherwise derivable from the present disclosures or known to those skilled in the art, can be provided as part of the kit. The components of the kit (e.g., displacement wraps 20, 120, introducers 40, 140) can be disposable such that they are for a single use and/or they can be used multiple times.

FIGS. 4A-4I illustrate one exemplary embodiment of a method of deploying the displacement wrap 120 at a surgical site 214 to move the tissue 212 from a first location 270 to a second location 272, referred to as a displacement location, shown in FIG. 4H. By way of non-limiting components, the displacement wrap 120 can be implemented as part of a shoulder reconstruction arthroplasty procedure. The displacement wrap 120 can be used to secure and move the tissue 212 (e.g., subscapularis tissue of the scapula) to provide access to a shoulder head 250. To the extent the present disclosure uses the displacement wrap 120 in illustrated embodiments, the use of the displacement wrap 120 is non-limiting, and other devices, including the displacement wrap 20 and other displacement wraps disclosed herein or otherwise derivable from the present disclosures, can be used and/or otherwise adapted for use in conjunction with the features and techniques of the present disclosures.

Referring to FIGS. 4A-4I, the tissue 212 can be prepared to have an accessible region for inserting the displacement wrap 120. The displacement wrap 120 includes the textile 122 and has the introducer 140 associated therewith, the introducer 140 being in the form of the grasper 146. As shown, a retractor 208 can be used to help move skin and other tissue to help permit access to the surgical site 214. Further, a person skilled in the art will recognize that various tools can be used to help prepare tissue 212 to allow for the introducer 140 to be inserted for use in disposing the displacement wrap 120 in a manner that allows it to be used to aid in providing access to the surgical site 214. For example, when the tissue 212 is the subscapularis muscle, tissue preparation can include, but is not limited to creation of an inferior access point that is inferior to the subscapularis muscle, creation of a superior opening through a rotator interval superior to the subscapularis muscle, and/or release of any capsular and/or glenohumeral attachments impeding access to placement of the displacement wrap 120 posterior to the subscapularis muscle. Tissue preparation can also include humeral head resection, humeral bone preparation, acetabular preparation, femoral head resection and/or broaching, open reduction and internal fixation (ORIF) for trauma plates to keep muscles from getting damaged, and/or insertion of any implant in the region, among others known to those skilled in the art. Although the illustrated method uses the displacement wrap 120 described above, at least some of the components associated with or otherwise used in conjunction with the device 120 may not be easily visible. In view of the disclosures provided for herein, and their related illustrations, a person skilled in the art will understand how the various components of the displacement wrap 120 engage the various parts of the patient's anatomy, and/or the portions of the displacement wrap 120 and other surgical tools tool(s) used in conjunction with the procedures disclosed with respect to FIGS. 4A-4I.

The introducer 140, as shown the grasper 146, can be guided or otherwise directed such that it is disposed under the tissue 212. The grasper 146 can be passed inferiorly or superiorly behind the tissue 212. For example, the grasper 146 can pass through an inferior region 252 and into a superior region 254. In some procedures, it can be beneficial to move the tissue 212 to both the inferior region 252 and the superior region 254 during the course of the procedure. A person skilled in the art will recognize the direction and force required to pass the grasper 146 through regions 252, 254. Other tools, including but not limited to forceps or clamps (not shown), can be used to pass the grasper 146 through regions 252, 254. Following the passage of the grasper 146, the first portion 122a, which is attached to the grasper 146, can be passed or slid through the regions 252, 254. Sliding the first portion 122a through the regions 252, 244 allows the textile 122 to contact the tissue 212. As shown in FIG. 4E, the grasper 146 can then be passed through the locking opening 130 of the second portion 122b.

Referring to FIGS. 4F-4I, the displacement wrap 120 can be tightened around the tissue 212 by adjusting the tension being applied to the tissue 212. The introducer 140 can be used as a handle to tighten the textile 122 around the tissue 212 after one or more of the locking features 124 is passed through the locking opening 130 and engages a portion of the textile 122 adjacent to and/or that defines the locking opening 130 to put the displacement wrap 120 in a first locked position. The user can access the introducer 140 either directly or via one or more tools. The introducer 140 can be further used to move the tissue 212 in a non-limiting direction to allow the user access to the shoulder head 250. For example, as shown the user can move the tissue 212 in an inferior direction from the first location 270, as shown in FIGS. 4A and 4G, to the second location 272, as shown in FIG. 4H. Any part of the introducer 140 can be clamped, fixed, or attached to any external object being used during the surgery (e.g., surgical drape) to prevent the displacement wrap 120 from dislodging from a desired location. For example, the grasper 146 can be attached or clamped to a surgical drape.

The user can move the tissue 212 from the first location 270 to the second location 272 without adjusting the displacement wrap 120 and while keeping the displacement wrap 120 in the first locked position. Alternatively, or additionally, the amount of tension on the tissue 212 can be adjusted, maintained, and/or altered by the user before, during, or after surgery. The user can unlock the locking feature(s) 124 from the first locked position, adjust the amount of tension on the tissue 212, for instance by moving the first portion 122a of the textile 122 with respect to the second portion 122b of the textile 122, and engaging one or more of the locking features 124 to place the displacement wrap 120 in a second (or additional) locked position. By moving the tissue 212 to the second location 272, it can enable better access to the surgical site 214, thus allowing surgeons to more easily perform actions such as, by way of non-limiting examples, more accurate humeral cuts, glenoid preparation for various shoulder procedures, and access to an implant or a location where an implant is to be disposed. After the procedure(s) are completed, the displacement wrap 120 can be disengaged from the tissue 212 and the tissue 212 can return to its normal, e.g., first location 270, and it can be in a condition substantially the same as it was prior to the start of the procedure because of the non-traumatic engagement of the displacement wrap 120. In some embodiments, the locking feature(s) 124 can be unlocked and relocked to move or secure the displacement wrap 120 more than one time during surgery. The locking feature(s) 124 can be variably adjusted for tissue 212 tightening and/or loosening. This is true for any of the locking features provided for herein or otherwise derivable from the present disclosures.

While the surgical procedure description provided with respect to FIGS. 4A-4I is directed to use of the displacement wrap 120 in conjunction with manipulating a subscapularis tissue, a person skilled in the art, in view of the present disclosures, will appreciate a variety of surgical procedures with which the displacement wrap 120 and/or other displacement wraps provided for herein or otherwise derivable from the present disclosures can be used. This includes, but is not limited to procedures that involve making humeral cuts, preparing a glenoid preparation for various shoulder procedures, various hip procedures, and/or accessing an implant or a location where an implant is to be disposed.

FIGS. 5A and 5B illustrate alternative embodiments of displacement wraps 220, 320 that utilize a locking suture 230, 330, to assist in engaging locking features 224, 324 of the displacement wraps 220, 320, respectively. As shown in FIG. 5A, the displacement wrap 220 includes a textile 222 similar to the textile 122 of FIG. 2, although an introducer 240 associated with the displacement wrap 220 comprises a suture 242 rather than rigid grasper as it is with respect to the textile 122. The textile 222 differs in one respect from the textile 122 because it does not include a locking opening 130. Instead, a locking suture 230 is coupled to a second portion 222b of the textile 222, such as by passing the locking suture 230 through the textile and forming one or more knots therein. As shown, a sliding knot 232 can be formed in the locking suture 230, creating an adjustable loop 234, which defines an opening, that can have its diameter adjusted by moving the sliding knot 232 along the suture 230. For example, a suture tail 236 of the suture 230 can have tension applied thereto, as shown in a direction F, to collapse a diameter of the adjustable loop 234. The loop 234 of the locking suture 230 can be used to engage slit(s) 226 of the locking features 224 formed on a first portion 222a of the textile, and the sliding knot 232 can be moved to draw the first portion 222a of the textile 222 towards the second portion 222b of the textile 222. The size of the loop 234 can be, at least temporarily, held in place to place the displacement wrap 220 in a locked position, allowing the displacement wrap 220, and tissue associated therewith, as shown tissue 312, to be secured at a desired tension and/or compression and/or at a desired location. More generally, a person skilled in the art, in view of the present disclosures, will understand how the locking suture 230 can be tightened using techniques provided for herein or otherwise known to those skilled in the art to place the displacement wrap 220 in a locked position based on a slit(s) 226 of the locking features 224 being engaged by the locking suture 230.

The displacement wrap 320 of FIG. 5B also incorporates a locking suture, as shown the locking suture 330. The displacement wrap 320 is similar to that of the displacement wrap 220, although one difference is that an introducer 340 associated with the same comprises a grasper 346, akin to the grasper 146 of FIG. 2. As shown, the locking suture 330 can operate similar to the locking suture 230 of FIG. 5A, using a sliding knot 332 to form an adjustable loop 334, which defines an opening, that can be controlled by a suture tail 336. Further, the locking suture 330 can be coupled to a second portion 322b of a textile 322 of the displacement wrap 320 and the locking suture 330 can engage one or more slits 326 that are a locking feature(s) 324 formed in a first portion 322a of the textile 322. Accordingly, the locking suture 330 can place the displacement wrap 320 in one or more locked positions as provided for herein.

FIG. 5C illustrates an alternative way by which the locking suture 330 can be placed in a locked position. As shown, the suture 330 can be passed into a hinge, such as a living hinge 338, which can be operated to secure the suture 330, and with it a position of the second portion 322b of the textile 322 with respect to the first portion 322a of the textile 322 (the textile 322 and related portions 322a, 322b being illustrated in FIG. 5B). Alternatively, the textile 322 can be molded into the introducer 340 without using a suture loop 148 shown in FIG. 2. Such a textile 322 molded to the introducer 340 can be secured, for example, by passing it through the living hinge 338. The living hinge 338 can then be snap shut to retain the textile 322 in position.

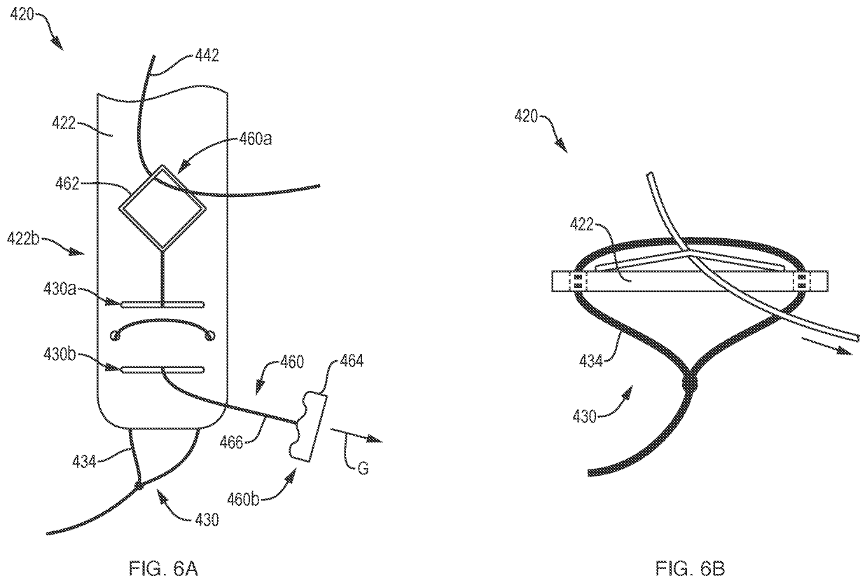

FIGS. 6A and 6B illustrate one non-limiting embodiment for associating a displacement wrap with a locking suture, as illustrated a displacement wrap 420 that comprises a textile 422 and a locking suture 430. In the illustrated embodiment, a plurality of openings, as shown slots 430a, 430b, are formed in a second portion 422b of the textile 422, and the locking suture 430 is disposed between the slots 430a, 430b. An attachment mechanism, as shown a kite wire 460, can be associated with the plurality of openings 430a, 430b and can be used to draw the textile 422 into a loop 434 of the locking suture 430. More specifically, the kite wire 460 can include a first end 460a having an opening 462 formed by a wire, a second end 460b having a handle or grasping portion 464, and an intermediate portion 466, as shown a wire that can be used to form the opening 462, although it can be a separate component from the wire in other embodiments. The opening 462 can be configured to receive the textile 422 therein and the handle 464 can be used to manipulate the intermediate portion 466 and the first end 460a, and thus the textile 422 associated therewith. As shown in FIG. 6A, the opening 462 is disposed on one side of the slots 430a, 430b, the intermediate portion 466, is passed through the slot 430a, under the textile 422, and out of the other slot 430b, and the handle 464 is disposed on an opposite side of the slots 430a, 430b.

More specifically, in the illustrated embodiment, an introducer 440, shown as a suture 442, can be passed into the opening 462. The suture 442 can pull a first portion (not shown) of the textile 422 into and through the opening 462. As a result, when a force G is applied to the handle 464 as shown in FIG. 6A, the opening 462 of the kite wire 460 is pulled through the slots 430a, 430b, in turn also pulling the suture 442 and the first portion of the textile 422 through slots 430a, 430b. Because the locking suture 430 is disposed between the slots 430a, 430b as shown, in turn the first portion of the textile 422 becomes disposed in the loop 434 of the locking suture 430. Similar to other embodiments, as shown in FIG. 6B, tightening or otherwise collapsing the loop 434 can construct the first portion (not shown) of the textile 422 on the second portion 422b of the textile 422. FIG. 6B actually shows the suture 442 being drawn to the second portion 422b of the textile 422. A person skilled in the art will appreciate that the suture 442 can pull the first portion of the textile 422 into the desired location and/or the first portion of the textile 422 can be positioned in a similar manner as the illustrated suture 442.

FIGS. 7A, 7B, and 7C illustrate alternative embodiments of displacement wraps 520, 520′ that utilize a textile or body 522, 522′ having a mesh or net structure that includes a plurality of openings 521, 521′ formed therein by the mesh or net structure. As shown in FIG. 7A, the mesh or net structure can be formed, for example, by weaving one or more sutures 525, filament, or other similar material, among other materials or objects known to those skilled in the art for forming mesh or net structure to be used in the body, within the confines of the body 522. In FIGS. 7B and 7C, the body 522′ itself has the openings 521′ formed therein. Like other embodiments, the mesh or net structure can be coated with one or more materials. The mesh or net structure of the textile 522, 522′ de-bulks the displacement wraps 520, 520′ while still protecting tissue around which the wrap 520, 520′ is disposed and distributing the load around that tissue (as shown with respect to tissue 512′ in FIGS. 7B and 7C). Similar to other embodiments, a first portion 522a, 522a′ of the body 522, 522′ can have one or more locking features or structures 524, 524′ formed therein, as shown slits 526, 526′, and a second portion 522b, 522b′ of the body 522, 522′ can include a locking opening 530, 530′ formed therein. The slits 526, 526′ can engage a portion of the body 520, 520′ that defines the locking opening 530, 530′ to allow the wrap 520, 520′ to be selectively locked and unlocked to supply varying degrees of tension and/or to assist in allowing the wrap 520, 520′ to manipulate tissue as described herein.

In use, as shown in FIGS. 7B and 7C with respect to the displacement wrap 520′, an introducer 540′, shown as rigid grasper 546′, can be coupled to the first portion 522a′ of the textile 522′, such as by passing a suture 548′ through an opening 523′ formed in the first portion 522a′ of the textile 522′ and having that suture 548′ also coupled to the introducer 540′. As described elsewhere herein, the introducer 540′ can be used to position the displacement wrap 520′ behind tissue, as shown a subscapularis tissue 512′. A terminal end of the first portion 522a′ of the textile 522′ can be passed through the locking opening 530′ and s electively locked in place using the slits 526′ of the locking feature 524′. The net or mesh configuration of the textile 522′, including the openings 521′ formed therein, can provide for a de-bulked implant result, thus resulting in the low profile configuration illustrated in FIG. 7C. The displacement wraps 520, 522 can include the same types of configurations and/or operate in similar manners as described herein with respect to other disclosed embodiments and/or embodiments derivable from the present disclosure.

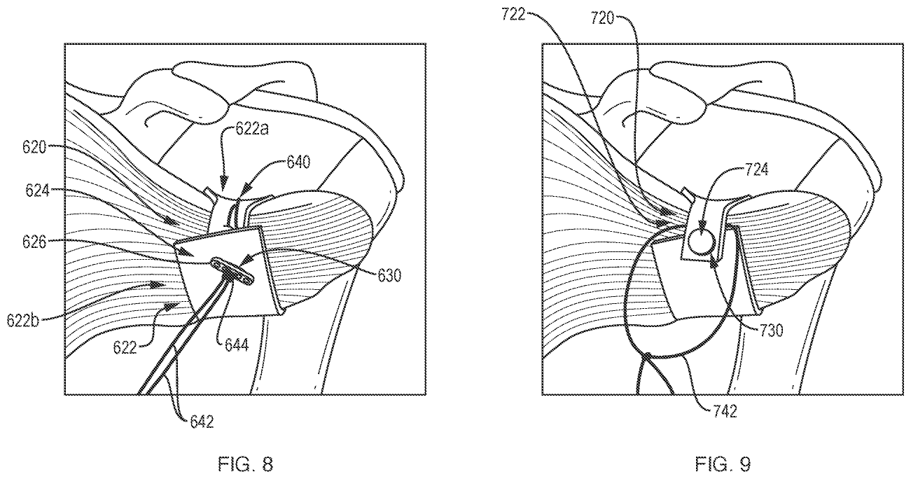

FIG. 8 illustrates an alternative embodiments of a displacement wrap 620 that comprises a textile or body 622 and a locking feature 624. The locking feature 624 can be a button 626, sometimes referred to as a tibial button or cortical button. Some non-limiting examples of cortical buttons are described in U.S. Pat. Nos. 9,757,113, 9,888,998, 9,974,643, and 10,405,968, the contents of each which is incorporated by reference herein in its entirety. By way of non-limiting example, in the illustrated embodiment, the cortical button 626 can be attached to an introducer 640, shown as a suture 642 having a sliding knot 644 formed therein such that the suture 642 forms a loop. The introducer 640 can be coupled to a first portion 622a of the body 622, using any number of techniques provided for herein or otherwise known to those skilled in the art. The suture 642 and the cortical button 626 can be passed through a locking opening 630 formed in a second portion 622b of the body 622, with the cortical button 626 engaging a portion of the body 622 that defines, and/or is proximate to the portion of the body 622 that defines, the locking opening 630, similar to the way slits (e.g., slits 126) of a locking feature (e.g., locking feature 124) engage a portion of a body (e.g., body 122) that defines a locking opening (e.g., locking opening 130) in other embodiments (e.g., the displacement wrap 120 of FIGS. 2-3C). The sliding knot 644 formed in the suture 642, can be moved to adjust a size of the loop formed by the suture 642, thus allowing tension to be increased and eased as desired in conjunction with manipulating the displacement wrap 620.

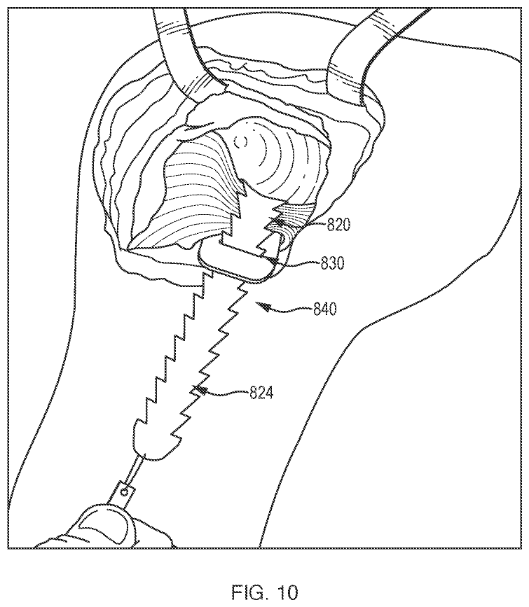

FIG. 9 illustrates an alternative embodiment of a displacement wrap 720 that comprises a textile 722 and a locking feature 724. The locking feature 724 can be a button head 726 that can be mated with or passed through a locking opening 730 to position the displacement wrap 720. A suture 742 can be used to attach and/or secure the displacement wrap 720 to an external object being used during surgery. A person skilled in the art, in view of the present disclosures, will appreciate that the displacement wrap 720 can include more than one button head 726 along the textile 722 to enable the displacement wrap 720 to be tightened to varying degrees and/or more than one locking opening 730 for similar purposes. The relatedness of this disclosure as compared to other embodiments of displacement wraps provided for herein means a further description of each of the components of the displacement wrap 720 is unnecessary.

FIG. 10 illustrates an alternative embodiments of a displacement wrap 820 that comprises a 3D-printed zip tie structure 840 that includes locking features 824 that can pass through a locking opening 830. The locking features 824 can be 3D-printed along one or, as shown, two edges of the zip tie 840. The displacement wrap 820 can be entirely comprised of plastic or any other material that can be molded, welded, and/or 3D-printed, including but not limited to as scaffold-type biologic, other biologics, and/or various materials provided for herein. The displacement wrap 820 can be 3D-printed as a single piece component as shown in FIG. 10. Alternatively, the displacement wrap 820 can be 3D-printed as separate components that can be combined, welded, or otherwise attached. The relatedness of this disclosure as compared to other embodiments of displacement wraps provided for herein means a further description of each of the components of the displacement wrap 820 is unnecessary.

As discussed herein, in at least some instances the displacement wraps and introducers disclosed herein can be designed to be disposed of after a single use, and/or in at least some instances the displacement wraps and introducers disclosed herein can be designed to be used multiple times. In either case, however, the devices and introducers can be reconditioned for reuse after at least one use. Reconditioning can include any combination of the steps of disassembly of the devices and/or introducers, for instance disassembling them from each other, followed by cleaning or replacement of particular pieces, and subsequent reassembly where appropriate. Upon cleaning and/or replacement of particular parts, the devices and introducers can be reassembled for subsequent use either at a reconditioning facility, or by a surgical team immediately prior to a surgical procedure. Those skilled in the art will appreciate that reconditioning of displacement wraps and/or introducers can utilize a variety of techniques for disassembly, cleaning/replacement, and reassembly. Use of such techniques, and the resulting reconditioned displacement wraps and/or introducers, are all within the scope of the present disclosure.

The displacement wraps and introducers described herein can be provided in a sterilized state for single patient use and/or otherwise be processed before surgery. First, a new or used displacement wrap and/or introducer is obtained and if necessary cleaned. The displacement wrap and/or introducer can then be sterilized. In one sterilization technique, the displacement wrap and/or introducer is placed in a closed and sealed container, such as a plastic or TYVEK bag. The container and displacement wrap and/or introducer are then placed in a field of radiation that can penetrate the container, such as gamma radiation, x-rays, or high-energy electrons. The radiation kills bacteria on the displacement wrap and/or introducer and in the container. The sterilized displacement wrap and/or introducer can then be stored in the sterile container. The sealed container keeps the displacement wrap and/or introducer sterile until it is opened in the medical facility.

It is preferred that the displacement wrap and/or introducer is sterilized. This can be done by any number of ways known to those skilled in the art including beta or gamma radiation, ethylene oxide, steam, and a liquid bath (e.g., cold soak).

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

1. A surgical method, comprising:
disposing a displacement wrap around at least a portion of a tissue;
placing the displacement wrap in a locked position to hold the tissue in at least one of tension or compression;
moving the displacement wrap to a displacement location to cause the tissue to move away from its original location to a new location, thereby increasing at least one of access to a surgical site or an available region behind the tissue for a surgical procedure to be performed; and
maintaining the displacement wrap at the displacement location, and thus the tissue at the new location, to allow access to at least one of the surgical site or the available region behind the tissue during the surgical procedure,
wherein the displacement wrap protects the tissue and distributes load across the displacement wrap and the tissue, thus preventing damage to the tissue.

2. The method of claim 1, wherein the displacement wrap comprises:
a first portion that includes a first terminal end and a textile; and
a second portion that includes a second, opposed terminal end, the second, opposed terminal end being coupled to an introducer.

3. The method of claim 2, further comprising:
passing the introducer under the tissue and subsequently sliding the wrap with respect to the tissue to move the textile into contact with the tissue,
wherein disposing a displacement wrap around at least a portion of a tissue further comprises wrapping the textile around at least a portion of the tissue, and
wherein the textile is wrapped around at least a portion of the tissue when the displacement wrap is in the locked position.

4. (canceled)

5. The method of claim 2, wherein placing the displacement wrap in a locked position further comprises:
passing the introducer and a first end of the textile coupled to the introducer through an opening formed in a second end of the textile; and
engaging at least one locking feature of the first end of the textile with a portion of the second end of the textile, proximate to the opening, to hold the tissue in at least one of tension or compression.

6. The method of claim 1, wherein moving the displacement wrap to a displacement location further comprises moving the tissue both superiorly and inferiorly during the surgical procedure without adjusting a positioning of the displacement wrap with respect to the tissue.

7. The method of claim 1, wherein the tissue is a subscapularis tendon.

8. The method of claim 1, further comprising:
unlocking the displacement wrap from the locked position, the locked position being a first locked position;
adjusting an amount of at least one of tension or compression being applied to the tissue; and
placing the displacement wrap in a second locked position,
wherein the second locked position is one of a same locked position or a different locked position than the first locked position.

9. The method of claim 8, wherein adjusting an amount of at least one of tension or compression being applied to the tissue further comprises:
sliding a portion of the displacement wrap with respect to itself,
wherein in the first locked position, a first locking feature on a first end of the displacement wrap engages a second end of the displacement wrap, and
wherein in the second locked position, a second locking feature on the first end of the displacement wrap engages the second end of the displacement wrap.

10. The method of claim 1, wherein maintaining the displacement wrap at the displacement location further comprises:
holding the tissue in at least one of tension or compression; and
attaching a portion of the displacement wrap to an object to maintain the at least one of tension or compression at the displacement location.

11. The method of claim 1, wherein placing the displacement wrap in a locked position to hold the tissue in at least one of tension or compression further comprises:
passing a first portion of the displacement wrap through a loop formed by a locking suture that is coupled to the displacement wrap;
engaging at least one locking feature of the first portion of the displacement wrap with the locking suture to hold the tissue in tension; and
collapsing the loop against the first portion of the displacement wrap to place the displacement wrap in the locked position.

12. A device for manipulating tissue during surgery, comprising:
a textile having a body with a length and a width, the length being greater than the width, the body comprising:
a first portion having a plurality of locking features disposed along the length of the textile;
a second portion having a locking opening formed therein and a width of the second portion being greater than a width of the first portion, the widths of the first and second portions being disposed in a same orientation as the width of the textile,
wherein the locking opening of the second portion is configured to receive the first portion such that a tissue receiving opening is formed by at least one of the first and second portions and each locking feature of the plurality of locking features is configured to engage with the second portion, proximate to the locking opening, to place the device in a locked configuration in which the location of the first portion is maintained with respect to the second portion and tissue disposed in the tissue-receiving opening is able to be secured within the tissue-receiving opening, and
wherein the textile is configured to protect the tissue it engages and distribute load across the textile and the tissue, thus preventing damage to the tissue.

13. (canceled)

14. The device of claim 12, wherein the plurality of locking features comprise:
a plurality of first diagonal slits formed along a length of a first side of the first portion of the body, the slits of the plurality of first diagonal slits being substantially parallel to each other; and
a plurality of second diagonal slits formed along a length of a second side of the first portion of the body, the slits of the plurality of second diagonal slits being substantially parallel to each other, the second side of the first portion of the body being opposed to the first side of the first portion of the body and the length of the first and second sides of the first portion of the body being disposed in a same orientation as the length of the textile.

15. The device of claim 12, wherein the textile comprises at least one of cotton, nylon, polyester, silk, poly felt, silicone, plastic, rubber, rubber-like materials, biologics, scaffold-type biologics, sutures, filaments, or a combination thereof.

16. (canceled)

17. The device of claim 12, wherein the body further comprises at least one of a mesh or net structure having a plurality of holes formed therein.

18. The device of claim 12, further comprising:
an introducer configured to pass into the locking opening of the second portion of the body.

19. The device of claim 18, wherein the introducer is integrally formed with the textile.

20. The device of claim 18, wherein the introducer is coupled to the textile.

21. A device for manipulating tissue during surgery, comprising:
a textile having a body with a length and a width, the length being greater than the width, the body comprising:
a first portion having a plurality of locking features disposed along the length of the textile;
a second portion having a locking suture coupled thereto and a width of the second portion being greater than a width of the first portion, the widths of the first and second portions being disposed in a same orientation as the width of the textile,
wherein the locking suture is configured to engage one or more locking features of the plurality of locking features to form a tissue receiving opening comprised of at least one of the first and second portions and to place the device in a locked configuration in which the location of the first portion is maintained with respect to the second portion and tissue disposed in the tissue-receiving opening is able to be secured within the tissue-receiving opening, and
wherein the textile is configured to protect the tissue it engages and distribute load across the textile and the tissue, thus preventing damage to the tissue.

22. The device of claim 21, wherein the plurality of locking features comprise:
a plurality of first diagonal slits formed along a length of a first side of the first portion of the body, the slits of the plurality of first diagonal slits being substantially parallel to each other; and
a plurality of second diagonal slits formed along a length of a second side of the first portion of the body, the slits of the plurality of second diagonal slits being substantially parallel to each other, the second side of the first portion of the body being opposed to the first side of the first portion of the body and the length of the first and second sides of the first portion of the body being disposed in a same orientation as the length of the textile.

23-27. (canceled)
